(19) United States
(12) Patent Application Publication
(10) Pub. No.: US 2003/0004424 A1
(43) Pub. Date: Jan. 2, 2003

(54) Heart rate monitor with a multiple exercise profile feature and method of using same

(76) Inventors: Burton H. Birnbaum, Woodmere, NY (US); Pertti Puolakanaho, Oulu (FI); Outi Hyyppa, Oulu (FI)

Correspondence Address: HOFFMANN & BARON, LLP, 6900 JERICHO TURNPIKE, SYOSSET, NY, 11791, US

(21) Appl. No.: 09/894,999

(22) Filed: Jun. 28, 2001

(51) Int. Cl. A61B 5/04

(52) U.S. Cl. 600/520

(57) ABSTRACT

A heart rate monitor and methods for monitoring a user's heart rate, which enable the monitor to be used under different sets of exercise and physiological parameters or profiles without reprogramming between different profiles. The exercise parameters include an exercise type, a day of the week, a time, a user's name, a level of difficulty, and physiological parameters of the user including height, weight, age, and level of fitness. A sensor detects the user's heartbeat, and an input device enables the user to enter information to be associated with each of a plurality of exercise profiles. A memory device stores the profiles, and an indicating device provides a sensory indication of when the user is outside a target heart rate zone. The profiles may be uploaded from a computer and information obtained during the profiles may be downloaded for additional review, analysis, and processing.

### FIELD OF THE INVENTION

The present invention relates generally to a heart rate monitor and more particularly to an ambulatory heart rate monitor having multiple exercise profiles that may be selected in response to different exercise and/or physiological parameters.

### BACKGROUND OF THE INVENTION

A user's heart rate during exercise is measured with a heart rate monitor (HRM) in terms of the number of heartbeats that occur during a unit of time e.g., beats per minute. The HRM typically includes a chest unit that detects an electrical signal generated by the heart and displays the measured heart rate on a display unit. The display unit can additionally display indicators, such as predetermined heart rate limits or target zones.

Conventional ambulatory HRMs provide a single profile to guide a user during an exercise routine having a single set of parameters. For instance, if the user is running, the HRM can be programmed to indicate when the user's heart rate exceeds predetermined upper and lower limits during a jogging routine. These limits can also be programmed to be different at different times during the profile, such as lower during warm-up and cool-down portions and higher during an intermediate portion of the profile. These portions may be further subdivided into laps, sprints, and the like, which substantially complicates the process of programming the HRM.

Entry of information associated with the profile is further complicated if the user performs different types of exercise routines requiring substantially different profiles, such as weightlifting, cycling, rowing, spinning, and the like. Programming of the HRM is even more complex if the user wants use and retain profiles having different intensities for the same activity under different circumstances, such as the day of the week and various physiological parameters. In either of these instances, the user is forced to reprogram the HRM with a different profile.

Such a task is both time-consuming and impractical, particularly for users who alternate activities on a daily basis. The need to reprogram the HRM each day following an exercise routine for that particular day typically discourages the majority of users from performing each of their exercise routines with the HRM. This significantly limits the effectiveness of the HRM as a tool for maintaining the user's level of fitness.

### OBJECTS AND SUMMARY OF THE INVENTION

It is an object of the present invention to provide an ambulatory heart rate monitor having multiple exercise profiles that may be used to monitor a user's heart rate or to guide a user in his/her training during different exercise routines and activities.

It is a further object of the present invention to provide a heart rate monitor, which avoids the necessity of reprogramming a heart rate monitor with different exercise profiles each time the user performs a different activity or exercise routine.

It is still a further object of the present invention to provide a heart rate monitor that is capable of inputting a plurality of pre-programmed exercise profiles.

It is another object of the present invention to provide a heart rate monitor, which is simpler to program and requires less time to initialize prior to its use.

It is still another object of the present invention to provide a heart rate monitor that is able to support multiple exercise profiles tailored to the physiological parameters and activities of a particular user.

It is an object of the present invention to provide a heart rate monitor that is able to record heart rate information of a user during multiple exercise routines for downloading, processing, and analysis by a computer.

It is another object of the present invention to provide a heart rate monitor that is able to upload pre-programmed exercise profiles from a computer and download heart rate information collected during a user's exercise routine to the computer for further review, processing, and analysis via a bidirectional wired or wireless link, such as an infrared and/or telemetric link.

These links make it possible to upload a pre-programmed plurality of exercise profiles from the computer 50 to the receiver 18 and upload It is an object of the present invention to provide an ambulatory method of monitoring a user's heart rate, which supports multiple exercise profiles that may be used during different exercise routines and activities.

It is a further object of the present invention to provide a method of monitoring a user's heart rate, which avoids the necessity of reprogramming a heart rate monitor with different exercise profiles each time the user performs a different activity or exercise routine.

It is still a further object of the present invention to provide a method for monitoring a user's heart rate that is capable of inputting a plurality of preprogrammed exercise profiles.

It is another object of the present invention to provide a method for monitoring a user's heart rate, which substantially simplifies the process of programming a heart rate monitor and reduces the amount of time required by the user to initialize the monitor prior to its use.

It is still another object of the present invention to provide a method for monitoring a user's heart rate that is able to support multiple exercise profiles tailored to the physiological parameters and activities of a particular user.

It is an object of the present invention to provide a method for monitoring a user's heart rate that is capable of recording heart rate information during multiple exercise routines for downloading, processing, and analysis by a computer.

The present invention provides an apparatus for monitoring a user's heart rate, which includes a sensor, an input device, memory, an indicating device, and a processing device. The sensor detects the user's heart rate, and an exercise profile program is stored in memory. The program enables one of a plurality of exercise profiles, which are also stored in memory, to be selected and information to be associated therewith. The processing device is responsive to the sensor, input device, and a selected profile stored in memory. The indicating device is responsive to the processing device and the processing device stores and/or recalls information associated with each selected profile. The exercise parameter may include an exercise type, a day of the week, a time, a user's name, a level of difficulty, and a physiological parameter, such as height, weight, age, and level of fitness.

The present invention also provides a method of programming a heart rate monitor including the steps of selecting one of a plurality of profiles stored in memory, entering an upper heart rate limit and a lower heart rate limit to be associated with the selected profile stored in memory, and repeating these steps for at least one more of the profiles stored in memory.

The present invention further provides a method of monitoring a heart rate including the steps of selecting one of a plurality of profiles stored in memory, detecting the heart rate, comparing the detected heart rate to at least one of an upper heart rate limit and a lower heart rate limit associated with the selected profile, indicating whether the detected heart rate is at least one of greater than the upper heart rate limit and less than the lower heart rate limit associated with the selected profile, and repeating these steps for at least one more of the profiles stored in memory.

A preferred embodiment of a heart rate monitor and corresponding methods of use, as well as other features and advantages of this invention, will be apparent from the following detailed description, which is to be read in conjunction with the accompanying drawings.

### BRIEF DESCRIPTION OF THE DRAWINGS

FIG. 1 shows a person using a heart rate monitor formed in accordance with the present invention.

FIG. 2 is a top view of an electrode belt of the heart rate monitor shown in FIG. 1.

FIG. 3 is a block diagram of the components of the heart rate monitor formed in accordance with the present invention.

FIG. 4 is a block diagram of the receiver in the heart rate monitor shown in FIG. 3.

FIG. 5 is a flowchart showing the top-level functions of the heart rate monitor formed in accordance with the present invention.

FIGS. 6A and 6B is a flowchart showing the top-level function of programming multiple profiles, as first shown in FIG. 5.

FIGS. 7A and 7B is a flowchart showing the top-level function of reviewing heart rate information, as first shown in FIG. 5.

### DETAILED DESCRIPTION OF THE PREFERRED EMBODIMENT

The heart rate monitor formed in accordance with the present invention is a completely ambulatory device that enables an individual to exercise while being guided by a plurality of exercise profiles, at home, at a health club, or anywhere else. The heart rate monitor includes a sensor, which is preferably located on the user's chest, which detects the user's heartbeat from an electrical signal generated by the heart.

The monitor also displays a heart rate, which is calculated from the number of heartbeats that occur during a given interval of time. Such heart rate monitors are intended to include chest units that are wired to a display, chest units that wirelessly transmit heart rate data to a receiver and a display in a wrist unit, and heart rate monitors that operate solely on the wrist without a chest transmitter.

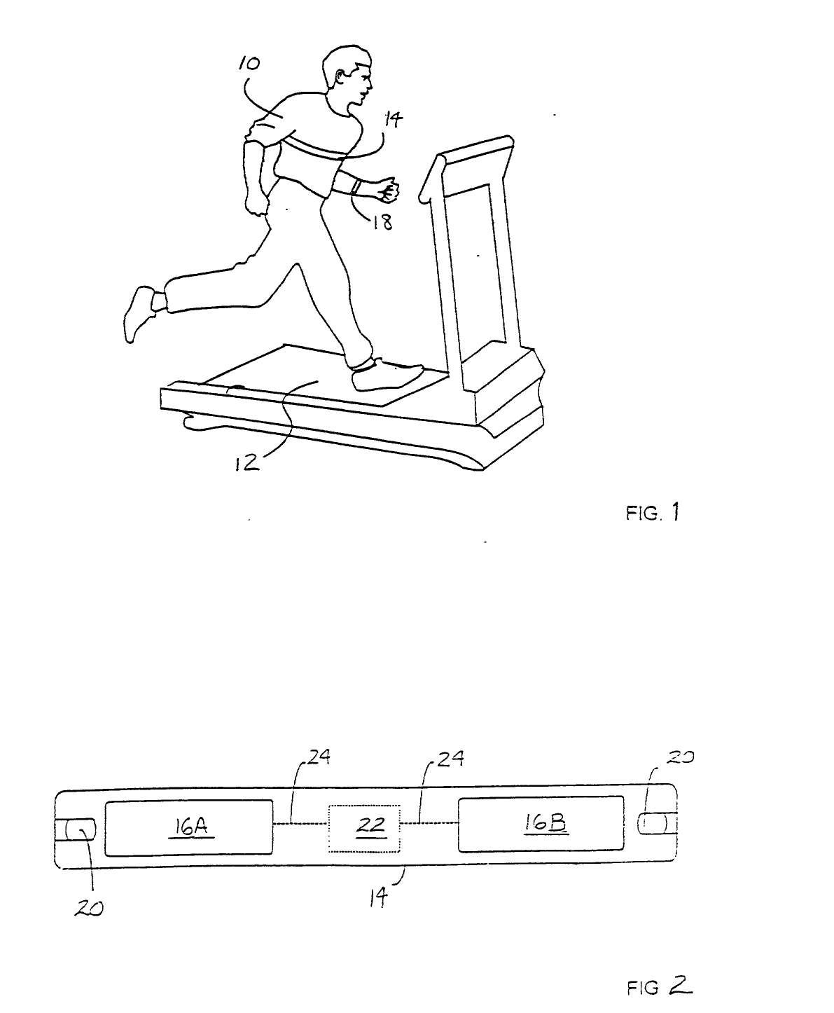

FIG. 1 shows a user 10 running on a treadmill 12. The heartbeat of the user 10 is detected by a transmitter electrode belt 14, which is preferably arranged on the user's chest. The heartbeat is detected with two or more electrodes 16A and 16B provided on the transmitter electrode belt 14. An electrical potential is generated between the electrodes in response to the beats. The transmitter electrode belt 14 is attached around the user's body with, for instance, a band made of an elastic material. A signal representing the detected heartbeat is transmitted preferably inductively to a receiver 18 on the wrist, which preferably also includes a display for indicating the heart rate, such as beats per minute.

As described above, the heart rate monitor may also be a wrist device, in which the transmitter and receiver are integrated in one device. The heartbeat may be detected by the wrist unit from either an electrocardiogram (ECG) signal, which is derived from two electrodes (e.g., a wrist electrode in contact with one of the user's arms and a finger electrode in contact with another of the user's arms), from an arterial pressure pulse, or by observing optically detectable changes in blood flow.

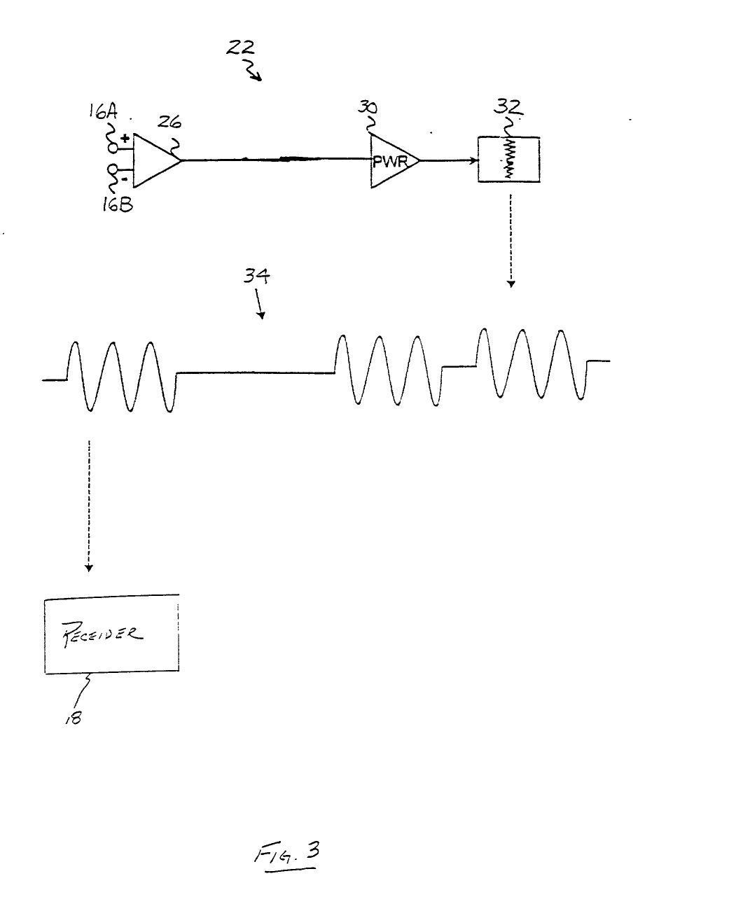

The electrode belt 14 is shown in greater detail in FIG. 2, which shows the electrode belt 14 from the side of the electrodes 16A and 16B, i.e. from the side facing the body of the user. FIG. 2 also shows fasteners 20 that attach the electrode belt 14 to the elastic band, which is strapped around the body. A dotted line in FIG. 2 represents an electronics unit 22 for processing the heart rate information received from the electrodes 16A and 16B. The electrodes 16A and 16B are electrically connected to the electronics unit 22 by conductive lines 24.

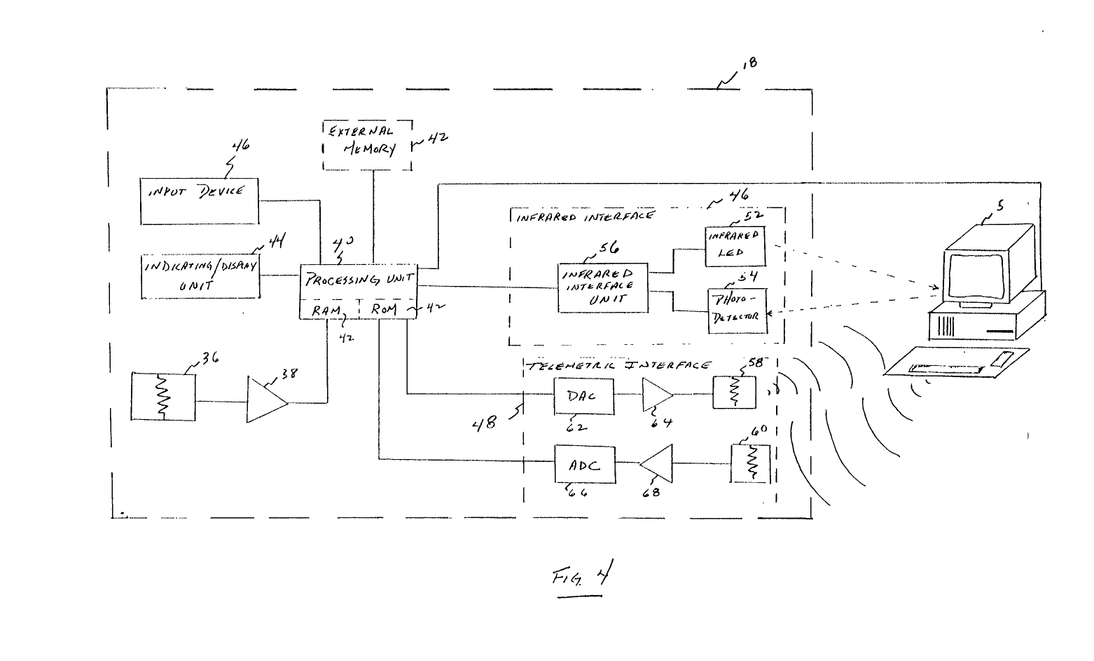

FIG. 3 is a block diagram of the preferred embodiments of the electronics unit 22 on the transmitter electrode belt 14 and a receiver 18, which is preferably worn on the user's wrist. The electronics unit 22 is shown at the top of the figure, a sample of heartbeat information to be transmitted is shown in the middle of the figure, and a block representing the receiver unit 18 is shown at the bottom of the figure. The electronics unit 22 receives the heartbeat information from the electrodes 16A and 16B. Alternatively, heart rate information may be processed in the electronics unit 22 of the transmitter 14 and transmitted as a heart rate value to the receiver 18.

From the electrodes, a heart rate signal is supplied to an amplifier 26 from which the signal is outputted to a power amplifier 30 and then to a transmitter 32. The transmitter 32 is preferably implemented as a coil, which inductively transmits heartbeat information 34 to the receiver, such as the receiver unit 18 to be arranged on the user's wrist or to, for instance, an external computer.

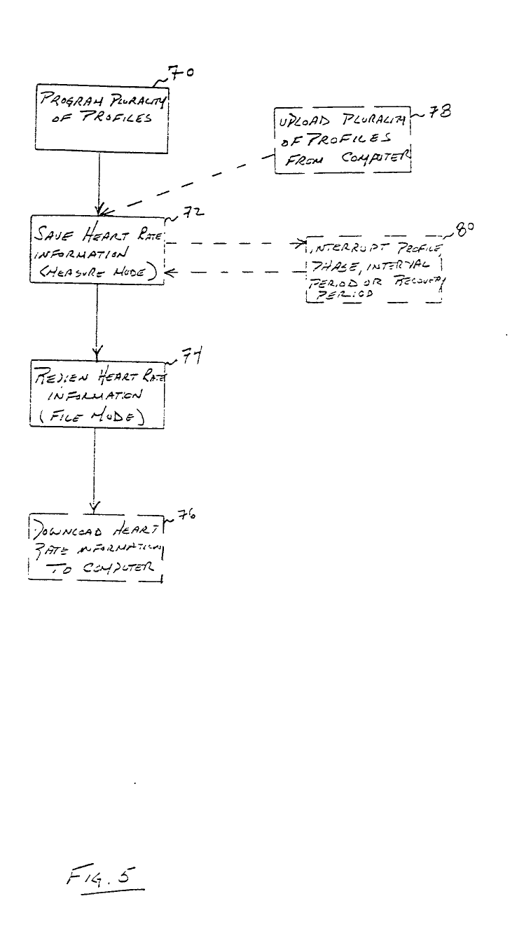

The heartbeat information 34 may be transmitted inductively, optically, through a wired conductor, or alternative means well known in the art. In one embodiment shown in FIG. 4, the receiver 18, such as the receiver to be worn on the wrist, includes a receiver coil 36 from which the received signal is outputted to a processing unit 40, such as a microcontroller or microprocessor, via a signal receiver or amplifier 38. The processing unit 40 controls and coordinates the operation of the different elements of the receiver 18.

The receiver 18 includes a memory device 42, which may be internal or external to the processing unit 40, for storing heartbeat information. The receiver 18 also includes an indicating device and display 44 for displaying the heart rate and other variables and visually and/or audibly indicating when the user is outside a target heart rate zone.

The receiver 18 includes an input device 46, such as one or more selectable buttons, a keypad, an electronic notepad, or a speech control means. The input device 46 is used to activate the monitor and to initiate different functional modes of the monitor. The input device 46 of the present invention further enables the user to electronically enter or program information to be associated with a plurality of exercise profiles, such as heart rate limits and the duration of specified portions of the exercise profile, such as phases, interval periods, and recovery periods. The input device further preferably allows the user to electronically enter one or more exercise parameters (such as the day of the week, time, fitness level, exercise intensity, and the like) or physiological parameters unique to the user (such as weight, height, age, sex, self-reported exercise frequency, and the like).

A phase refers to a specific portion of the duration of an exercise profile selected from the plurality of exercise profiles. A plurality of interval periods and recovery periods preferably exist during each phase. The interval period refers to the duration of time during which the user is substantially exercising and the recovery period refers to the duration of time during which the user is substantially at rest.

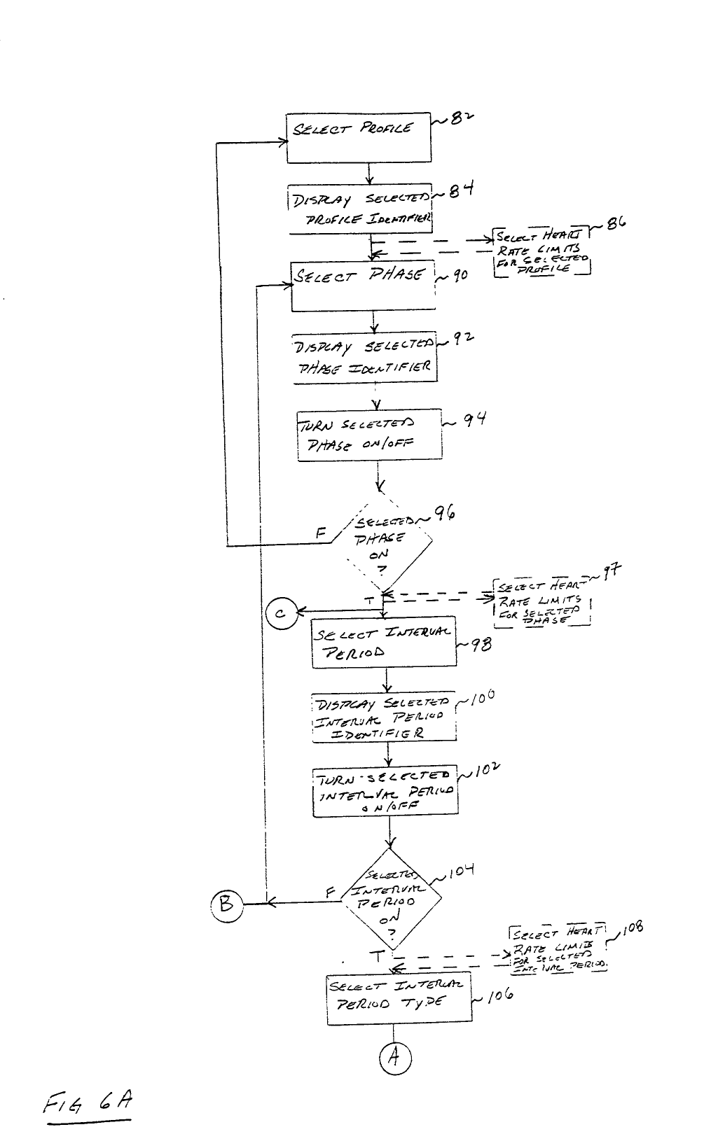

The receiver 18 further includes the processing unit 40, which performs a multiple exercise profile program. The method used by the program will be described in detail with reference to FIGS. 5-7 below. In the preferred embodiment, the program is implemented in software using a general-purpose microprocessor or microcontroller. However, the method may also be implemented in an application specific integrated circuit (ASIC), by discrete logic components, or by employing alternative means well known in the art.

An infrared interface 46 and a telemetric interface 48 preferably provide bi-directional communication links between the receiver 18 and a computer, such as a personal computer. The telemetric interface 48 may include an inductive interface and/or an audio interface. These links make it possible to upload a pre-programmed plurality of exercise profiles from the computer 50 to the receiver 18 and download heart rate information collected during the user's exercise routines to the computer 50 for further review, processing, and analysis.

The infrared interface 46 preferably includes an infrared light emitting diode (LED) 52, which translates an electrical signal into pulses of infrared light and transmits these pulses from the receiver 18 to the computer 50. The infrared interface 46 also preferably includes a photo-detector 54, which receives infrared light pulses from the computer 50 and translates these pulses into an electrical signal. An infrared interface unit 56 is coupled to the infrared LED 52 and photo-detector 54 and performs the electrical translations necessary to interface these components 52, 54 with the processing unit 40.

The telemetric interface 48 preferably includes a coil 58, which translates an electrical signal into a magnetic signal and transmits the magnetic signal from the receiver 18 to the computer 50. The telemetric interface 48 may also include a coil 60, which receives a magnetic signal from the computer 50 and translates the magnetic signal into an electrical signal. A digital-to-analog converter 62 and an amplifier 64 translate the digital signal from the processing unit 40 to an analog signal suitable for the coil 58. An analog-to-digital converter 66 and an amplifier 68 translate the analog signal provided by the coil 60 to a digital signal suitable for the processing unit 40. Such wireless and telemetric data transmission techniques have been described in U.S. Pat. Nos. 6,229,454; 5,690,119; and U.S. application Ser. No. 09/716,630, which are incorporated herein by reference.

FIG. 5 is a flowchart showing the top-level functions of the heart rate monitor formed in accordance with the present invention. The plurality of exercise profiles are programmed manually in step 70. While the user is exercising and the heart rate monitor is in a measure mode, heart rate information is stored in the heart rate monitor in step 72.

It must be noted that the subject invention is able to guide the user in maintaining his heart rate within a target zone while performing a plurality of exercise routines. The heart rate monitor formed in accordance with the present invention accomplishes this by being able to store, revise, and manipulate a plurality of exercise profiles. Each of the exercise profiles may be tailored to a particular set of exercise or physiological parameters, thereby eliminating the need for the user to reprogram the heart rate monitor with a different exercise profile when switching between routines.

The user may optionally review the heart rate information while in file mode in step 74, and may optionally download the heart rate information to a computer for further processing and analysis in step 76. As discussed above, the user may upload a pre-programmed plurality of profiles from a computer in step 78 rather than manually programming the plurality of profiles in step 70. The user may also interrupt a currently running profile, phase, interval period, or recovery period while in the measure mode in step 80.

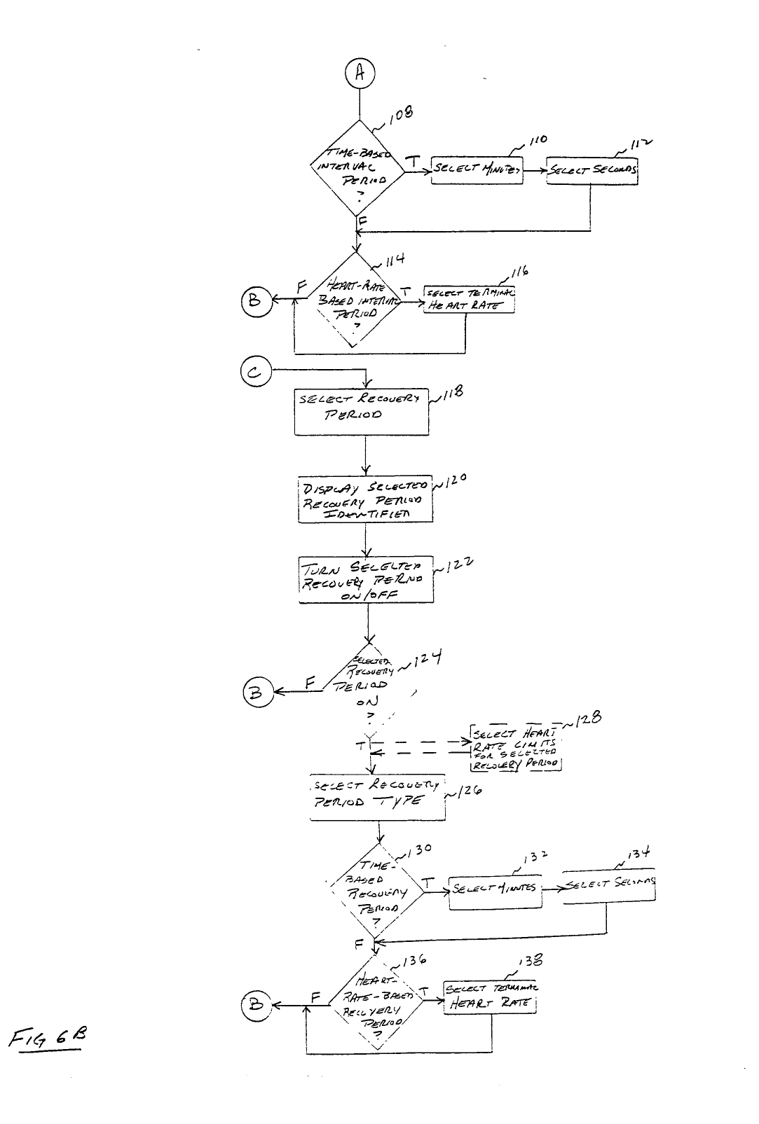

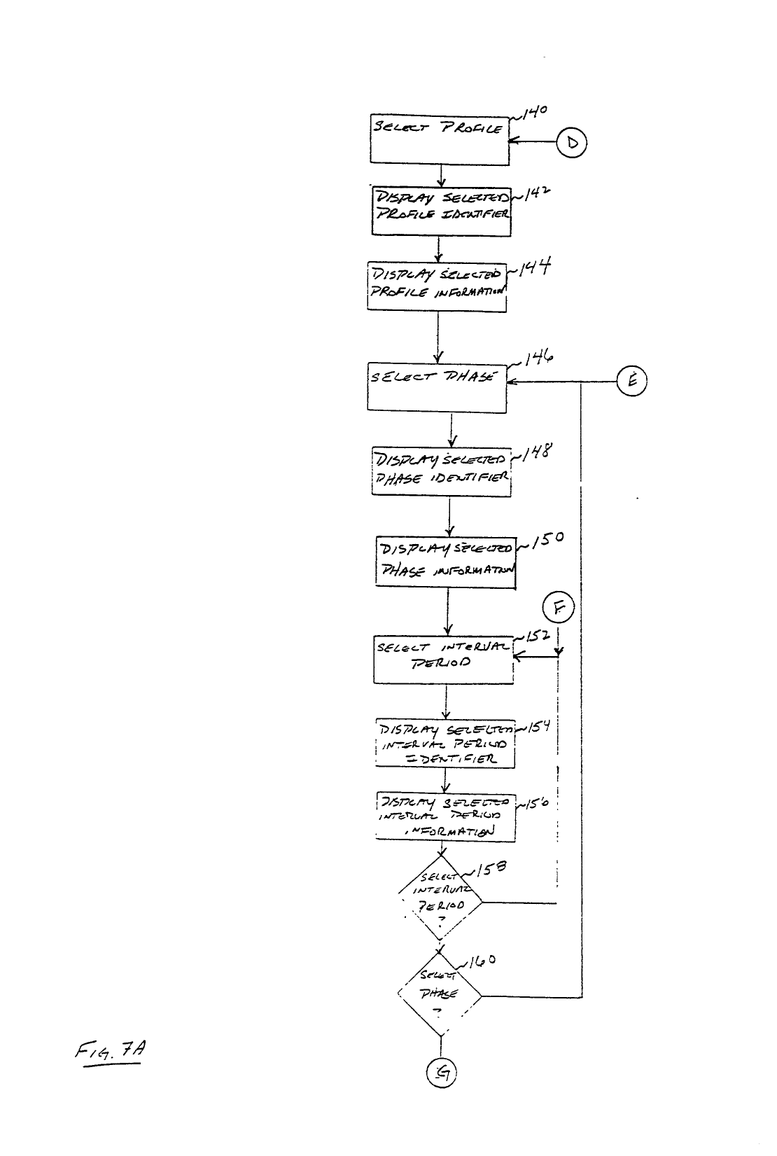

Step 70 in FIG. 5 is shown in greater detail in FIGS. 6A and 6B, which detail the function of manually programming the plurality of profiles. Each of the plurality of exercise profiles is selected in step 82 and the selected profile identifier is preferably displayed in step 84. The profile identifier may be modified by the user or retained as a numerical identifier. Heart rate limits or a target heart rate zone may be selected by the user for the selected profile in step 86.

A phase in the selected profile is then selected in step 90, and the selected phase identifier is displayed in step 92. Like the profile identifier, the phase identifier may optionally be modified by the user or retained as a numerical identifier. The user may then choose to turn the selected phase on or off in step 94. As described above with respect to the profile, the user may set heart rate limits or a target heart rate zone for the selected phase in step 97. If the user chooses to turn the selected phase off, the user may then select the same or another profile by returning to step 82 via step 96.

If the selected phase is determined to be turned on in step 96, the user will then select an interval period within the selected phase in step 98, and the selected interval period identifier will be displayed in step 100. As described above, the user may modify the interval period identifier or choose to retain a numerical identification of the selected interval period. The user can then choose to turn the selected interval on or off in step 102, and if it is determined that the selected interval period is on in step 104, the type of interval period may be selected in step 106. As with the selected profile and selected phase, the user may select heart rate limits or a target heart rate zone for the selected interval period in step 108. If the user has chosen to turn the selected interval off in step 102, the user may then select the same or another phase in step 90 via step 104.

The interval period types include manual, time-rate-based, and heart-rate-based. The manual interval period terminates in response to the user selecting a button on the heart rate monitor. The time-rate-based interval period terminates after a specified duration of time, and the heart-rate-based interval period terminates when a specified terminal heart rate has been achieved.

If the user selects a time-based interval period in step 108 of FIG. 6B, the user will then select the minutes in step 110 and the seconds in step 112, which determine the duration of the selected interval period. If the user selects a heart-rate-based interval period in step 114, the user will select a terminal heart rate in step 116, which will terminate the selected interval period. The method then proceeds to step 90 to select the same or another phase.

Following the determination that the selected phase is turned on in step 96 of FIG. 6A, the user may select a recovery period within the selected profile and phase in step 118 of FIG. 6B. The selected recovery period identifier is then displayed in step 120, and the user may choose to turn the selected recovery period on or off in step 122. As with the profile, phase, and interval period, the recovery period identifier may be modified by the user or retained as a numerical identifier. If the selected recovery period has been determined to be on in step 124, the user will select the recovery period type in step 126. The user may select heart rate limits or a target heart rate zone for the selected recovery period as a means of implementing a biofeedback approach to induce relaxation between interval periods in step 128.

The recovery period may be time-based or heart-rate-based. The time-based recovery period terminates following a selected time period and the heart-rate-based recovery period terminates in response to a terminal heart rate being achieved. If the user selects a time-based recovery period in step 130, the user will then select the minutes in step 132 and the seconds in step 134 after which the selected recovery period will terminate. If the user selects a heart-rate-based recovery period in step 136, the user will then select a terminal heart rate in step 138. The method will then return to select the same or another phase in step 90.

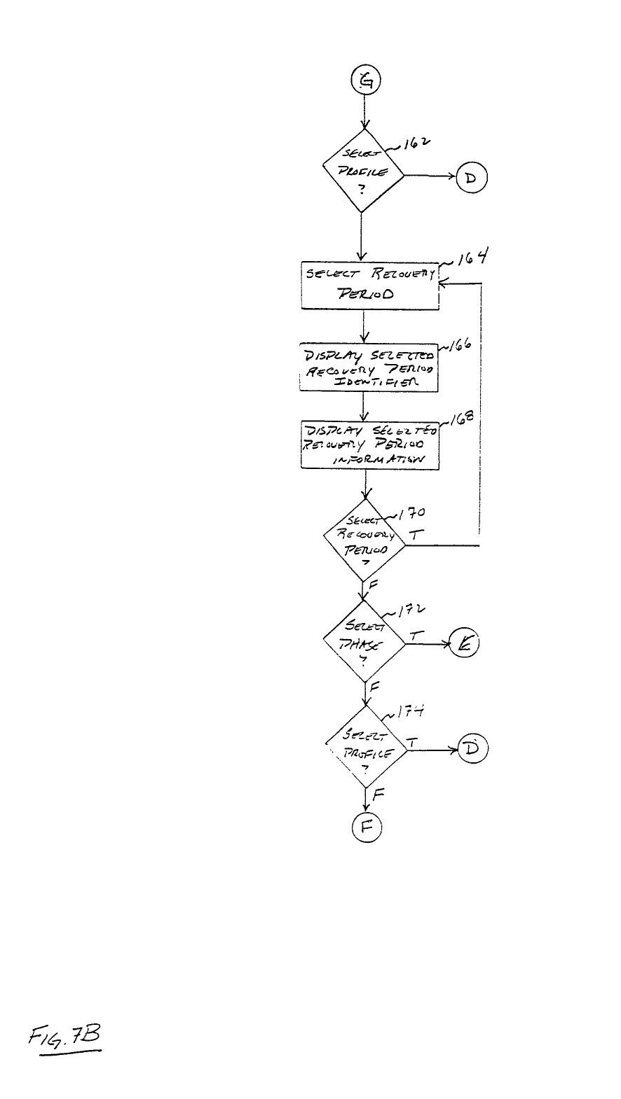

FIGS. 7A and 7B are a flowchart showing details of the function of reviewing heart rate information stored during a plurality of exercise profiles as shown in step 74 in FIG. 5. The user selects each of the plurality of profiles in step 140, and the selected profile identifier is displayed in step 142. The selected profile information, such as the start time, duration, target heart rate zone, average heart rate, maximum heart rate, and minimum heart rate is displayed in step 144. The user then selects the phase within the selected profile in step 146, and the phase identifier is displayed in step 148. Information concerning the selected phase, such as the start time, duration, target heart rate zone, average heart rate, maximum heart rate, and minimum heart rate is displayed in step 150.

The interval period in the selected phase and selected profile is then selected in step 152, and the selected interval period identifier is displayed in step 154. Information concerning the selected interval period, such as the start time, duration, target heart rate zone, average heart rate, maximum heart rate, and minimum heart rate is displayed in step 156. If the user would like to select the same or another interval period in step 158, the method returns to step 152, and if the user would like to select the same or another phase in step 160, the method returns to step 146. If the user would like to select the same or another profile in step 162 of FIG. 7B, the algorithm returns to step 140.

If none of these conditions are true, the algorithm continues with selecting a recovery period within the selected phase and selected profile in step 164. The recovery period identifier is displayed in step 166. Information concerning the selected recovery period is displayed in step 168, such as the start time, duration, target heart rate zone, average heart rate, maximum heart rate, minimum heart rate, drop in heart rate, and the time to attain the pre-set heart rate limit in heart-rate-based recovery periods.

If the user would like to select the same or another recovery period in step 170, the method returns to step 164, and if the user would like to select the same or another phase in step 172 the method returns to step 146. Finally, if the user would like to select the same or another profile in step 174, the method returns to step 140, and if none of the above are true, the method returns to select another interval period in step 152.

The invention thus provides an ambulatory heart monitor and method having multiple exercise profiles that may be used to monitor the user's heart rate during different exercise routines and activities without the necessity of reprogramming the monitor with different exercise profiles each time the user performs a different activity or exercise routine. The subject invention further provides a heart rate monitor and method that are capable of inputting a plurality of pre-programmed exercise profiles, which substantially simplify the process of programming the monitor and reduce the amount of time required by the user to initialize the monitor prior to its use.

In addition, the subject invention provides a heart monitor and method that are able to record the heart rate information of a user during multiple exercise routines for subsequent, downloading, processing, and analysis by a computer and is able to perform bidirectional communication with a computer via wired and wireless means, such as infrared and telemetric links.

Although the illustrative embodiments of the present invention have been described herein with reference to the accompanying drawings, it is to be understood that the invention is not limited to those precise embodiments and that various other changes and modifications may be effected therein by one skilled in the art without departing from the scope or spirit of the invention.

What is claimed is:

1. A heart rate monitor for monitoring a user's heart rate comprising:
at least one sensor for detecting the user's heart rate;
at least one input device;
a memory device having an exercise profile program stored therein, the exercise profile program enabling the selection of one of a plurality of profiles stored in the memory device, the exercise profile program enabling the storage of information to be associated with each selected profile, each of the plurality of profiles being associated with at least one exercise parameter;
at least one indicating device; and
a processing device, the processing device being responsive to the at least one sensor, the at least one input device, and a selected profile stored in the memory device, the at least one indicating device being responsive to the processing device, the processing device performing at least one of storing and recalling the information associated with each selected profile.

2. The heart rate monitor as defined by claim 1, wherein the at least one exercise parameter includes at least one of an exercise type, a day of the week, a time, a user's name, a level of difficulty, and a physiological parameter.

3. The heart rate monitor as defined by claim 2, wherein the physiological parameter includes at least one of height, weight, age, and level of fitness.

4. The heart rate monitor as defined by claim 1, wherein at least one of the plurality of profiles includes a plurality of phases, the duration of each of the plurality of phases being at least a portion of the duration of at least one of the plurality of profiles.

5. A heart rate monitor as defined by claim 4, wherein at least one of the plurality of phases includes at least one of an upper heart rate limit and a lower heart rate limit associated therewith, the indicating device indicating whether the detected heart rate is at least one of greater than the upper heart rate limit and less than the lower heart rate limit associated with the at least one of the plurality of profiles.

6. The heart rate monitor as defined by claim 4, wherein at least one of the plurality of phases includes at least one of an interval period and a recovery period, the interval period and the recovery period being representative of at least a portion of at least one of the plurality of phases, the interval period being associated with a period of time during which the user is substantially exercising, the recovery period being associated with a period of time during which the user is substantially at rest.

7. A heart rate monitor as defined by claim 6, wherein at least one of the interval period and the recovery period includes at least one of an upper heart rate limit and a lower heart rate limit associated therewith, the indicating device indicating whether the detected heart rate is at least one of greater than the upper heart rate limit and less than the lower heart rate limit associated with the at least one of the interval period and the recovery period.

8. The heart rate monitor as defined by claim 6, wherein information input by the user associates the interval period with at least one of a maximum duration and a terminal heart rate, the interval period terminating in response to attaining at least one of the maximum duration and the terminal heart rate

9. The heart rate monitor as defined by claim 6, wherein information input by the user associates the recovery period with at least one of a maximum duration and a terminal heart rate, the recovery period terminating in response to attaining at least one of the maximum duration and the terminal heart rate.

10. The heart rate monitor as defined by claim 1, wherein the at least one indicating device indicates at least one of the user's heart rate, programmable limits for the user's heart rate, and a duration of at least a portion of the selected profile.

11. The heart rate monitor as defined by claim 1, further comprising an infrared interface, the infrared interface providing an infrared communication link between the heart rate monitor and a computer.

12. The heart rate monitor as defined by claim 11, wherein the infrared interface includes an amplifier and an infrared light emitting diode.

13. The heart rate monitor as defined by claim 1, further comprising a telemetric interface, the telemetric interface providing a telemetric communication link between the heart rate monitor and a computer, the telemetric communication link including at least one of an inductive link and an audio link.

14. The heart rate monitor as defined by claim 13, wherein the telemetric interface includes an analog-to-digital converter, an amplifier, and a coil.

15. The heart rate monitor as defined by claim 1, wherein the information to be associated with each selected profile is input as a preprogrammed plurality of profiles.

16. The heart rate monitor as defined by claim 15, wherein the preprogrammed plurality of profiles is uploaded from a computer.

17. The heart rate monitor as defined by claim 15, wherein the preprogrammed plurality of profiles is output from an exercise profile application program, the exercise profile application program inputting at least one of an exercise parameter and a physiological parameter.

18. A method of programming a heart rate monitor comprising the steps of:
selecting one of a plurality of profiles stored in memory, each of the plurality of profiles being associated with at least one exercise parameter;
entering at least one of an upper heart rate limit and a lower heart rate limit to be associated with the selected profile and stored in memory; and
repeating the above steps for at least one more of the profiles stored in memory.

19. A method of programming a heart rate monitor as defined by claim 18, further comprising the step of storing into memory a unique profile identifier to be associated with each selected profile, the profile identifier being representative of at least one of an exercise type, a day of the week, a time, a user's name, a level of difficulty, and a physiological parameter.

20. A method of programming a heart rate monitor as defined by claim 19, wherein the physiological parameter includes at least one of height, weight, age, and level of fitness.

21. A method of programming a heart rate monitor as defined by claim 19, further comprising the step of displaying the profile identifier.

22. A method of programming a heart rate monitor as defined by claim 18, further comprising the step of selecting one of a plurality of phases, the selected phase being representative of at least a portion of the duration of the selected profile.

23. A method of programming a heart rate monitor as defined by claim 22, wherein the selected phase includes a phase identifier associated therewith, the method including the step of displaying the phase identifier.

24. A method of programming a heart rate monitor as defined by claim 22, further comprising the step of entering at least one of an upper heart rate limit and a lower heart rate limit to be associated with the selected phase.

25. A method of programming a heart rate monitor as defined by claim 22, further comprising the step of selecting at least one of an interval period and a recovery period, the interval period and the recovery period being representative of at least a portion of the duration of the selected phase, the interval period being associated with a period of time during which a user is substantially exercising, the recovery period being associated with a period of time during which the user is substantially at rest.

26. A method of programming a heart rate monitor as defined by claim 25, wherein the interval period includes an interval period identifier associated therewith, the method including the step of displaying the interval period identifier.

27. A method of programming a heart rate monitor as defined by claim 25, wherein the recovery period includes an recovery period identifier associated therewith, the method including the step of displaying the recovery period identifier.

28. A method of programming a heart rate monitor as defined by claim 25, further comprising the step of entering at least one of an upper heart rate limit and a lower heart rate limit to be associated with the selected at least one of the interval period and the recovery period.

29. A method of programming a heart rate monitor as defined by claim 25, further comprising the step of entering at least one of a maximum duration and a terminal heart rate to be associated with the interval period, the interval period terminating in response to attaining at least one of the maximum duration and the terminal heart rate.

30. A method of programming a heart rate monitor as defined by claim 25, further comprising the step of entering at least one of a maximum duration and a terminal heart rate to be associated with the recovery period, the recovery period terminating in response to attaining at least one of the maximum duration and the terminal heart rate.

31. A method of programming a heart rate monitor as defined by claim 18, further comprising the step of displaying at least one of the user's heart rate, programmable limits for the user's heart rate, and a duration of at least a portion of the selected profile.

32. A method of programming a heart rate monitor as defined by claim 18, further comprising the step of entering information to be associated with each selected profile, the information including a preprogrammed plurality of profiles.

33. A method of programming a heart rate monitor as defined by claim 32, wherein the step of entering information includes the step of uploading the preprogrammed plurality of profiles from a computer.

34. A method of programming a heart rate monitor as defined by claim 32, wherein the step of entering information includes the step of inputting the preprogrammed plurality of profiles from an exercise profile application program, the exercise profile application program inputting at least one of an exercise parameter and a physiological parameter.

35. A method of monitoring a heart rate comprising the steps of:
selecting one of a plurality of profiles stored in memory, each of the plurality of profiles having at least one of an upper heart rate limit and a lower heart rate limit associated therewith;
detecting the heart rate;
comparing the detected heart rate to at least one of the upper heart rate limit and the lower heart rate limit associated with the selected profile;
indicating whether the detected heart rate is at least one of greater than the upper heart rate limit and less than the lower heart rate limit associated with the selected profile; and
repeating the above steps for at least one more of the profiles stored in memory.

36. A method of monitoring a heart rate as defined by claim 35, wherein entering a unique profile identifier associated with each selected profile, the profile identifier being at least one of an exercise type, a day of the week, a time of day, a user's name, a level of difficulty, and a physiological parameter.

37. A method of monitoring a heart rate monitor as defined by claim 36, wherein the physiological parameter includes at least one of height, weight, age, and level of fitness.

38. A method of monitoring a heart rate as defined by claim 36, further comprising the step of displaying the profile identifier.

39. A method of monitoring a heart rate as defined by claim 35, wherein at least one of the plurality of profiles includes a plurality of phases, at least one of the plurality of phases being representative of at least a portion of the duration of each of the selected profiles.

40. A method of monitoring a heart rate as defined by claim 39, wherein the at least one of the plurality of phases includes a phase identifier associated therewith, the method including the step of displaying the phase identifier.

41. A method of monitoring a heart rate as defined by claim 39, wherein the at least one of the plurality of phases includes at least one of an upper heart rate limit and a lower heart rate limit associated therewith, the method further including the step of indicating whether the detected heart rate is at least one of greater than the upper heart rate limit and less than the lower heart rate limit associated with the at least one of the plurality of phases.

42. A method of monitoring a heart rate as defined by claim 39, wherein the at least one of the plurality of phases includes at least one of an interval period and a recovery period, the interval period and the recovery period being representative of at least portions of the duration of the at least one of the plurality of phases, the interval period being associated with a period of time during which a user is substantially exercising, the recovery period being associated with a period of time during which the user is substantially at rest.

43. A method of monitoring a heart rate as defined by claim 42, wherein the interval period includes an interval period identifier associated therewith, the method including the step of displaying the interval period identifier.

44. A method of monitoring a heart rate as defined by claim 42, wherein the recovery period includes a recovery period identifier associated therewith, the method including the step of displaying the recovery period identifier.

45. A method of monitoring a heart rate as defined by claim 42, wherein at least one of the interval period and the recovery period include at least one of an upper heart rate limit and a lower heart rate limit associated therewith, the method including the step of indicating whether the detected heart rate is at least one of greater than the upper heart rate limit and less than the lower heart rate limit associated within the at least one of the interval period and the recovery period.

46. A method of monitoring a heart rate as defined by claim 42, wherein the interval period includes at least one of a maximum duration and a terminal heart rate associated therewith, the method including the step of terminating the interval period in response to attaining at least one of the maximum duration and the terminal heart rate.

47. A method of monitoring a heart rate as defined by claim 42, wherein the recovery period includes at least one of a maximum duration and a terminal heart rate associated therewith, the method including the step of terminating the recovery period in response to attaining at least one of the maximum duration and the terminal heart rate.

48. The method of monitoring a heart rate as defined by claim 35, further comprising the step of displaying at least one of the user's heart rate, programmable limits for the user's heart rate, and a duration of at least a portion of the selected profile.

49. The method of monitoring a heart rate as defined by claim 35, further comprising the step of storing the detected heart rate occurring during each of the selected profiles in memory.

50. The method of monitoring a heart rate as defined by claim 35, further comprising the step of downloading the detected heart rate occurring during each of the selected profiles via at least one of an infrared link and a telemetric link to a computer.

51. The method of monitoring a heart rate as defined by claim 50, further comprising the step of processing the downloaded heart rate occurring during each of the selected profiles.
